(19) United States
(12) Patent Application Publication
(10) Pub. No.: US 2015/0122283 A1
(43) Pub. Date: May 7, 2015

(54) COSMETIC HEAT-PROTECTION AGENT

(71) Applicant: Henkel AG & Co. KGaA, Duesseldorf (DE)

(72) Inventors: Thorsten Knappe, Schenefeld (DE); Helga Van Flodrop, Duesseldorf (DE)

(21) Appl. No.: 14/591,984

(22) Filed: Jan. 8, 2015

Related U.S. Application Data
(63) Continuation of application No. PCT/EP2013/062912, filed on Jun. 20, 2013.

(30) Foreign Application Priority Data
Jul. 10, 2012 (DE) 10 2012 212 010.6

(51) Int. Cl. A61K 8/65; A45D 7/02; A61Q 5/00; A61Q 5/04; A61K 8/41; A61K 8/81

(52) U.S. Cl. CPC A61K 8/65; A61K 8/416; A61K 8/8182; A61Q 5/00; A61Q 5/04; A45D 7/02; A61K 2800/594
USPC 132/202; 424/70.2; 424/70.14

(57) ABSTRACT

Cosmetic compositions containing a) at least one first quaternary ammonium compound from the group of quaternized collagen hydrolysates; b) at least one second quaternary ammonium compound which differs from a), c) at least one film-forming polymer, d) water are suitable as heat-protection agents in the thermally assisted temporary deformation of keratinic fibers.

### FIELD OF THE INVENTION

The present invention generally relates to the technical field of the temporary or permanent deformation of human hair, and more particularly relates to heat-protection agents for use in methods for thermally assisted temporary or permanent reshaping of human

### BACKGROUND OF THE INVENTION

A range of different techniques are used for permanently or temporarily shaping human hair and for example take the form of curling, straightening, back-combing or indeed setting. These techniques are based on the use of setting cosmetic agents, or “styling agents”, such as hair sprays, hair waxes, hair gels, hair setting preparations, setting lotions or styling sprays.

As an alternative or in addition to treatment with setting cosmetic agents, the hair may be subjected to thermal treatment.

Smooth hair is waved or curled by deforming the hair with exposure to heat, for example with the assistance of a hairdryer with diffuser or curling tongs. The smooth hair is here wound around the heated curling tongs or the diffuser with temperatures of up to 250° C. being achieved in the process. In this method, styling sprays which are applied to the hair before the actual heat treatment are usually used to improve the styling result.

Special hair straighteners are used for straightening hair. Hair straighteners have two parallel metal or ceramic plates between which the hair is drawn once the plates have heated up. Conventional commercial hair straighteners may be heated up to temperatures in the range from 150-250° C. The purpose of using hair straighteners is to straighten wavy to curly hair physically/using heat. If hair is to be straightened using hair straighteners, a styling spray, also known as straightening spray, is usually applied to the hair beforehand as a hair straightener styling agent. The spray helps the straighteners to slide and assists straightening of the hair.

Numerous cosmetic compositions for temporarily deforming hair, including for use with exposure to heat, are known from the prior art. European patent application EP 1 750 656 A1 describes heat protection sprays which, in addition to further ingredients, contain cationic wheat protein hydrolysates.

The disadvantage of the cosmetic compositions for temporarily deforming hair with exposure to heat which are known from the prior art is the intense odor they form on application of heat which can cause slight throat irritation to the user during heat treatment of the hair. Furthermore, the heat protection provided to the hair by these agents is in need of improvement.

It was thus an object of the present invention to provide a cosmetic composition for treating hair with exposure to heat which is stable even at temperatures of 250° C., releases no substances which are harmful to health at these elevated temperatures and, when used in this temperature range, exhibits odor formation which is distinctly reduced in comparison with conventional compositions. In particular in deformation methods in the temperature range above 150° C., the cosmetic composition should furthermore be distinguished by improved heat-protection characteristics and thus provide the hair with better protection from the harmful action of the elevated temperatures used than do conventional agents.

It has been found that the above-described technical objects may be achieved by specific heat-protection sprays based on quaternized collagen hydrolysates. U.S. Pat. No. 4,374,125 and German patent application DE 41 09 999 A1 describe the use of collagen hydrolysates in hair care agents.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic composition containing a) at least one first quaternary ammonium compound from the group of quaternized collagen hydrolysates; b) at least one second quaternary ammonium compound which differs from a); c) at least one film-forming polymer; and d) water.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present application accordingly firstly provides a cosmetic composition containing
a) at least one first quaternary ammonium compound from the group of quaternized collagen hydrolysates;
b) at least one second quaternary ammonium compound which differs from a)
c) at least one film-forming polymer
d) water.

The cosmetic compositions according to the invention contain as first essential component a quaternary ammonium compound a) from the group of quaternized collagen hydrolysates. In preferred compositions, the proportion by weight of the quaternary ammonium compound a), which is also known as cationic derivatized collagen hydrolysates, in the total weight of the composition amounts to 0.05 to 4.0 wt. %, preferably 0.1 to 2.0 wt. % and in particular 0.2 to 1.0 wt. %.

Cationically derivatized collagen hydrolysates a) are mixtures of substances which are obtained for example by reacting proteins hydrolysed by alkaline, acidic or enzymatic methods with cationic alkylating agents. Such alkylating agents are for example glycidyltrialkylammonium chlorides or 3-chloro-2-hydroxypropyltrialkylammonium chlorides. These compounds preferably contain three methyl groups or a long-chain alkyl- or acylamidopropyl group and two methyl groups in the quaternary ammonium group. The cationically derivatized collagen hydrolysates a) are then obtained by condensation with the protein hydrolysates. Compounds which contain a long alkyl chain are preferred. Preferred cationically derivatized collagen hydrolysates a) have an N,N-dimethyl-N—C₈₋₂₂-alkyl-ammonium group, preferably an N,N-dimethyl-N—C₈₋₁₆-alkyl-ammonium group. Preferred cationically derivatized collagen hydrolysates a) are in particular characterized by an N,N-dimethyl-N-coconut alkyl-ammonium group. More preferred quaternized collagen hydrolysates are known under the INCI name Lauryldimonium Hydroxypropyl Hydrolyzed Collagen.

With regard to the cosmetic properties, in particular the heat protection, of the cosmetic compositions, it has proven advantageous to use quaternary ammonium compounds a) with a molecular weight of above 1000 g/mol, preferably between 1200 and 10000 g/mol, more preferably between 1500 and 5000 g/mol and in particular between 2000 and 2800 g/mol. Corresponding agents are preferred according to the invention.

The agents according to the invention contain as second essential component at least one second quaternary ammonium compound which differs from a). The proportion by weight of this quaternary ammonium compound b) in the total weight of the cosmetic composition preferably amounts to 0.1 to 6.0 wt. %, preferably 0.2 to 4.0 wt. % and in particular 0.4 to 2.0 wt. %.

With regard to the heat-protection action of cosmetic compositions according to the invention, it has proven advantageous for the quaternary ammonium compound b) to have a molecular weight of below 1000 g/mol, preferably between 200 and 800 g/mol, more preferably between 250 and 500 g/mol and in particular between 300 and 400 g/mol.

Cationic surfactants of the quaternary ammonium compound and esterquat type are particularly suitable as the quaternary ammonium compound b). Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryltrimethylammonium chloride, lauryldimethylbenzylammonium chloride and tricetylmethylammonium chloride, and the imidazolinium compounds known under the INCI names Quaternium-27 and Quaternium-83. The long alkyl chains of the above-stated surfactants preferably comprise 10 to 18 carbon atoms.

Esterquats are known substances which contain both at least one ester function and at least one quaternary ammonium group as a structural element. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines.

More preferred cosmetic compositions contain a quaternary ammonium compound b) selected from the group of alkyltrimethylammonium halides, dialkyldimethylammonium halides and trialkylmethylammonium halides, preferably from the group of alkyltrimethylammonium chlorides, wherein cetyltrimethylammonium chloride is more preferred. The proportion by weight of alkyltrimethylammonium halides, dialkyldimethylammonium halides and trialkylmethylammonium halides, preferably from the group of alkyltrimethylammonium chlorides, in particular of cetyltrimethylammonium chloride in the total weight of the composition according to the invention preferably amounts to 0.05 to 4.0 wt. %, preferably 0.1 to 2.0 wt. % and in particular 0.2 to 1.0 wt. %.

In order to improve the product properties of the cosmetic compositions according to the invention it has proven advantageous to maintain a specific weight ratio of the quaternary ammonium compounds a) and b). It is for this reason that cosmetic compositions in which the weight ratio of quaternary ammonium compound a) to quaternary ammonium compound b) amounts to 5:1 to 1:5, preferably 4:1 to 1:4, more preferably 3:1 to 1:3 and in particular 2:1 to 1:2, are preferred.

The cosmetic preparations according to the invention contain a film-forming polymer c) as a further component. The proportion by weight of this polymer c) in the total weight of the cosmetic preparation preferably amounts to 0.5 to 12 wt. %, preferably 1.0 to 10 wt. % and in particular 2.0 to 8.0 wt. %.

Nonionic or cationic polymers are more preferentially used as film-forming polymers c).

Suitable nonionic polymers are for example:
vinylpyrrolidone/vinyl ester copolymers, as are distributed for example under the tradename Luviskol® (BASF). Luviskol® VA 64 and Luviskol® VA 73, in each case vinylpyrrolidone/vinyl acetate copolymers, are preferred nonionic polymers.
cellulose ethers, such as hydroxypropylcellulose, hydroxyethylcellulose and methylhydroxypropylcellulose, as are distributed for example under the tradename Culminal® and Benecel® (AQUALON).
shellac.
polyvinylpyrrolidones, as are distributed for example under the tradename Luviskol® (BASF).
siloxanes. These siloxanes may be either water-soluble or water-insoluble. Both volatile and non-volatile siloxanes are suitable, wherein non-volatile siloxanes are taken to be those compounds whose boiling point at standard pressure is above 200° C. Preferred siloxanes are polydialkylsiloxanes, such as for example polydimethylsiloxane, polyalkylarylsiloxanes, such as for example polyphenylmethylsiloxane, ethoxylated polydialkylsiloxanes and polydialkylsiloxanes which contain amine and/or hydroxy groups.
glycosidically substituted silicones according to EP 0 612 759 B1.

The water-soluble, film-forming vinylpyrrolidone/vinyl acetate copolymer obtainable under the trade name PVP/VA W-635 (INCI name: VP/VA Copolymer) is, for example, preferred according to the invention.

The compositions according to the invention may contain temperature-stable cationic polymers. Cationic polymers should be taken to mean polymers which comprise groups in the main and/or side chain which may be “temporarily” or “permanently” cationic. Polymers which are designated “permanently cationic” according to the invention are those which, irrespective of the pH value of the agent, comprise a cationic group. As a rule, these are polymers which contain a quaternary nitrogen atom, for example in the form of an ammonium group. Preferred cationic groups are quaternary ammonium groups. Polymers which have proven particularly suitable are in particular those in which the quaternary ammonium group is attached via a C₁₋₄ hydrocarbon group to a main polymer chain synthesized from acrylic acid, methacrylic acid or the derivatives thereof.

Homopolymers of general formula (III),

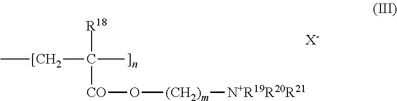

in which R¹⁸ is —H or —CH₃, R¹⁹, R²⁰ and R²¹ are mutually independently selected from C₁₋₄ alkyl, alkenyl or hydroxyalkyl groups, m=1, 2, 3 or 4, n is a natural number and X⁻ a physiologically acceptable organic or inorganic anion, and copolymers substantially consisting of the monomer units listed in formula (III) and nonionogenic monomer units are more preferred cationic polymers. In the context of these polymers, those which are preferred according to the invention are those for which at least one of the following conditions applies:
R¹⁸ denotes a methyl group
R¹⁹, R²⁰ and R²¹ denote methyl groups
m has the value 2.

Physiologically acceptable counterions X″ which may, for example, be considered are halide ions, sulfate ions, phosphate ions, methosulfate ions and organic ions such as lactate, citrate, tartrate and acetate ions. Halide ions, in particular chloride, are preferred.

A particularly suitable homopolymer is the optionally crosslinked poly(methacryloyloxyethyltrimethylammonium chloride) with the INCI name of Polyquaternium-37. Crosslinking may if desired proceed with the assistance of olefinically polyunsaturated compounds, for example divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylenebisacrylamide is a preferred crosslinking agent.

The homopolymer is preferably used in the form of a nonaqueous polymer dispersion which should have a polymer fraction of no less than 30 wt. %. Such polymer dispersions are commercially obtainable under the names Salcare® SC 95 (approx. 50% polymer fraction, further components: mineral oil (INCI name: Mineral Oil) and tridecyl-polyoxypropylene-polyoxyethylene ether (INCI name: PPG-1-Trideceth-6)) and Salcare® SC 96 (approx. 50% polymer fraction, further components: mixture of diesters of propylene glycol with a mixture of caprylic and capric acid (INCI name: Propylene Glycol Dicaprylate/Dicaprate) and tridecyl-polyoxypropylene-polyoxyethylene ether (INCI name: PPG-1-Trideceth-6)).

Copolymers with monomer units according to formula (III) preferably contain acrylamide, methacrylamide, acrylic acid C₁₋₄ alkyl esters and methacrylic acid C₁₋₄ alkyl esters as nonionogenic monomer units. Acrylamide is more preferred among these nonionogenic monomers. These copolymers, as described above for the homopolymers, may also be crosslinked. A copolymer which is preferred according to the invention is crosslinked acrylamide-methacryloyloxyethyltrimethylammonium chloride copolymer. Such copolymers, in which the monomers are present in a weight ratio of approximately 20:80, are commercially obtainable as approximately 50% nonaqueous polymer dispersions under the name Salcare® SC 92.

Further preferred cationic polymers are for example
quaternized cellulose derivatives, as are commercially obtainable under the names Celquat® and Polymer JR®. The compounds Celquat® H 100, Celquat® L 200 and Polymer JR®400 are preferred quaternized cellulose derivatives,
cationic alkyl polyglycosides according to patent DE 44 13 686,
cationized honey, for example the commercial product Honeyquat® 50,
cationic guar derivatives, such as in particular the products distributed under the trade names Cosmedia®Guar and Jaguar®,
polysiloxanes with quaternary groups, such as for example the commercially obtainable products Q2-7224 (manufacturer: Dow Corning; a stabilized trimethylsilylamodimethicone), Dow Corning® 929 Emulsion (containing a hydroxylamino-modified silicone which is also designated an amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxane, Quaternium-80).
polymeric dimethyldiallylammonium salts and the copolymers thereof with esters and amides of acrylic acid and methacrylic acid. The products commercially obtainable under the names Merquat®100 (poly(dimethyldiallylammonium chloride)) and Merquat®550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as for example vinylpyrrolidone-dimethylamino ethyl methacrylate copolymers quaternized with diethyl sulfate. Such compounds are commercially obtainable under the names Gafquat®734 and Gafquat®755,
vinylpyrrolidone-vinylimidazolium methochloride copolymers, as are offered for sale under the names Luviquat® FC 370, FC 550, FC 905 and HM 552 and Hold, quaternized polyvinyl alcohol.

Preferred cationic polymers are furthermore the polymers known under the INCI names
Polyquaternium-2,
Polyquaternium-17,
Polyquaternium-18 and
Polyquaternium-27
   with quaternary nitrogen atoms in the polymer main chain.

The polymers known under the names Polyquaternium-24 (commercial product, for example Quatrisoft® LM 200) may also be used as cationic polymers. Copolymers of vinylpyrrolidone, as are obtainable as commercial products Copolymer 845 (manufacturer: ISP), Gaffix® VC 713 (manufacturer: ISP), Gafquat®ASCP 1011, Gafquat®HS 110, Luviquat®8155 and Luviquat® MS 370 may likewise be used according to the invention.

“Temporarily cationic” polymers are further cationic polymers which are according to the invention. These polymers conventionally contain an amino group which at specific pH values assumes the form of a quaternary ammonium group and is thus cationic. Chitosan and the derivatives thereof, as are readily commercially available for example under the trade names Hydagen® CMF, Hydagen® HCMF, Kytamer® PC and Chitolam® NB/101, are for example preferred. Chitosans are deacetylated chitins which are commercially obtainable in various degrees of deacetylation and various degrees of degradation (molecular weights). The production thereof is described, for example, in DE 44 40 625 A1 and in DE 195 03 465 A1 Particularly highly suitable chitosans exhibit a degree of deacetylation of at least 80% and a molecular weight of 5·10⁵ to 5·10⁶ (g/mol).

In order to produce preparations according to the invention, the chitosan must be converted into the salt form. This may proceed by dissolution in dilute aqueous acids. Suitable acids are both mineral acids such as for example hydrochloric acid, sulfuric acid and phosphoric acid and organic acids, for example low molecular weight carboxylic acids, polycarboxylic acids and hydroxycarboxylic acids. Relatively high molecular weight alkylsulfonic acids or alkylsulfuric acids or organophosphoric acids may furthermore be used, provided that they have the necessary physiological acceptability. Suitable acids for converting the chitosans into the salt form are for example acetic acid, glycolic acid, tartaric acid, malic acid, citric acid, lactic acid, 2-pyrrolidinone-5-carboxylic acid, benzoic acid or salicylic acid. Low molecular weight hydroxycarboxylic acids such as for example glycolic acid or lactic acid are preferably used.

Preferred cationic, film-forming polymers are the vinylpyrrolidone/methoimidazolinium chloride copolymers as are offered for sale under the name “Luviquat®”, for example Luviquat FC 370 (INCI name: Polyquaternium-16), Luviquat Style (INCI name: Polyquaternium-16) and Luviquat Hold (INCI name: Polyquaternium-46), and the cationic terpolymer of vinylpyrrolidone, dimethylaminopropylmethacrylamide and lauryldimethylpropylmethacrylamidoammonium chloride known under the INCI name Polyquaternium-55 which is obtainable the name Styleze W.

To summarize, preferred cosmetic compositions are those in which the film-forming polymer c) is selected from the group of vinylpyrrolidone/methylimidazolinium chloride copolymers, vinylpyrrolidone/vinyl acetate copolymers and cationic terpolymers of vinylpyrrolidone, dimethylaminopropylmethacrylamide and lauryldimethylpropylmethacrylamidoammonium chloride, wherein vinylpyrrolidone/vinyl acetate copolymers are more preferred. The proportion by weight of this film-forming polymer c) from the group of vinylpyrrolidone/methylimidazolinium chloride copolymers, vinylpyrrolidone/vinyl acetate copolymers and cationic terpolymers of vinylpyrrolidone, dimethylaminopropylmethacrylamide and lauryldimethylpropylmethacrylamidoammonium chloride, in particular vinylpyrrolidone/vinyl acetate copolymers in the total weight of the cosmetic preparation preferably amounts to 0.5 to 12 wt. %, preferably 1.0 to 10 wt. % and in particular 2.0 to 8.0 wt. %.

The compositions according to the invention preferably contain water and/or ethanol as solvent. It is more preferred to use water as solvent. Cosmetic compositions which are characterized in that, relative to the total weight thereof, they contain 70 to 99 wt %, preferably 75 to 95 wt. % and in particular 80 to 90 wt. % water are preferred according to the invention. More preferred compositions contain water and ethanol, wherein the proportion by weight of ethanol is preferably distinctly below the proportion by weight of water and preferably amounts to 1.0 to 10 wt. %, more preferably 2.0 to 8.0 wt. % and in particular 3.0 to 6.0 wt. %, in each case relative to the total weight of the composition.

The pH value of compositions according to the invention preferably amounts to pH 4.0 to 8.0, preferably pH 5.0 to 7.5 and in particular pH 5.5 to 7.0.

The following tables show the composition of some preferred cosmetic agents (details in wt % relative to the total weight of the cosmetic agent unless otherwise stated).

| | Formula 1 | Formula 2 | Formula 3 | Formula 4 | Formula 5 |
|---|---|---|---|---|---|
| Quaternized collagen | 0.05 to 4.0 | 0.1 to 2.0 | 0.2 to 1.0 | 0.3 | 1.0 |
| hydrolysate a) | | | | | |
| Quaternary ammonium | 0.1 to 6.0 | 0.2 to 4.0 | 0.4 to 2.0 | 0.5 | 0.5 |
| compound b) | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Misc. | ad 100 | ad 100 | ad 100 | ad 100 | ad 100 |
| | | | | | Formula |
| | Formula 6 | Formula 7 | Formula 8 | Formula 9 | 10 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Quaternary ammonium | 0.1 to 6.0 | 0.2 to 4.0 | 0.4 to 2.0 | 0.5 | 0.5 |
| compound b) | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Misc. | ad 100 | ad 100 | ad 100 | ad 100 | ad 100 |
| | Formula | Formula | Formula | Formula | Formula |
| | 11 | 12 | 13 | 14 | 15 |
| Quaternized collagen | 0.05 to 4.0 | 0.1 to 2.0 | 0.2 to 1.0 | 0.3 | 1.0 |
| hydrolysate a) | | | | | |
| Cetyltrimethylammonium | 0.1 to 6.0 | 0.2 to 4.0 | 0.4 to 2.0 | 0.5 | 0.5 |
| chloride | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Misc. | ad 100 | ad 100 | ad 100 | ad 100 | ad 100 |
| | Formula | Formula | Formula | Formula | Formula |
| | 16 | 17 | 18 | 19 | 20 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Cetyltrimethylammonium | 0.1 to 6.0 | 0.2 to 4.0 | 0.4 to 2.0 | 0.5 | 0.5 |
| chloride | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Misc. | ad 100 | ad 100 | ad 100 | ad 100 | ad 100 |
| | Formula | Formula | Formula | Formula | Formula |
| | 21 | 22 | 23 | 24 | 25 |
| Quaternized collagen | 0.05 to 4.0 | 0.1 to 2.0 | 0.2 to 1.0 | 0.3 | 1.0 |
| hydrolysate a) | | | | | |
| Quaternary ammonium | 0.1 to 6.0 | 0.2 to 4.0 | 0.4 to 2.0 | 0.5 | 0.5 |
| compound b) | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Misc. | ad 100 | ad 100 | ad 100 | ad 100 | ad 100 |
| | Formula | Formula | Formula | Formula | Formula |
| | 26 | 27 | 28 | 29 | 30 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Quaternary ammonium | 0.1 to 6.0 | 0.2 to 4.0 | 0.4 to 2.0 | 0.5 | 0.5 |
| compound b) | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Misc. | ad 100 | ad 100 | ad 100 | ad 100 | ad 100 |
| | Formula | Formula | Formula | Formula | Formula |
| | 31 | 32 | 33 | 34 | 35 |
| Quaternized collagen | 0.05 to 4.0 | 0.1 to 2.0 | 0.2 to 1.0 | 0.3 | 1.0 |
| hydrolysate a) | | | | | |
| Cetyltrimethylammonium | 0.1 to 6.0 | 0.2 to 4.0 | 0.4 to 2.0 | 0.5 | 0.5 |
| chloride | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Misc. | ad 100 | ad 100 | ad 100 | ad 100 | ad 100 |
| | Formula | Formula | Formula | Formula | Formula |
| | 36 | 37 | 38 | 39 | 40 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Cetyltrimethylammonium | 0.1 to 6.0 | 0.2 to 4.0 | 0.4 to 2.0 | 0.5 | 0.5 |
| chloride | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Misc. | ad 100 | ad 100 | ad 100 | ad 100 | ad 100 |
| | Formula | Formula | Formula | Formula | Formula |
| | 41 | 42 | 43 | 44 | 45 |
| Quaternized collagen | 0.05 to 4.0 | 0.1 to 2.0 | 0.2 to 1.0 | 0.3 | 1.0 |
| hydrolysate a) | | | | | |
| Quaternary ammonium | 0.1 to 6.0 | 0.2 to 4.0 | 0.4 to 2.0 | 0.5 | 0.5 |
| compound b) | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 46 | 47 | 48 | 49 | 50 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Quaternary ammonium | 0.1 to 6.0 | 0.2 to 4.0 | 0.4 to 2.0 | 0.5 | 0.5 |
| compound b) | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 51 | 52 | 53 | 54 | 55 |
| Quaternized collagen | 0.05 to 4.0 | 0.1 to 2.0 | 0.2 to 1.0 | 0.3 | 1.0 |
| hydrolysate a) | | | | | |
| Cetyltrimethylammonium | 0.1 to 6.0 | 0.2 to 4.0 | 0.4 to 2.0 | 0.5 | 0.5 |
| chloride | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 56 | 57 | 58 | 59 | 60 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Cetyltrimethylammonium | 0.1 to 6.0 | 0.2 to 4.0 | 0.4 to 2.0 | 0.5 | 0.5 |
| chloride | | | | | |
| Film-forming polymer | 0.5 to 12 | 1.0 to 10 | 2.0 to 8.0 | 5.0 | 2.0 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 61 | 62 | 63 | 64 | 65 |
| Quaternized collagen | 0.05 to 4.0 | 0.1 to 2.0 | 0.2 to 1.0 | 0.3 | 1.0 |
| hydrolysate a) | | | | | |
| Quaternary ammonium | 0.1 to 6.0 | 0.2 to 4.0 | 0.4 to 2.0 | 0.5 | 0.5 |
| compound b) | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 66 | 67 | 68 | 69 | 70 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Quaternary ammonium | 0.1 to 6.0 | 0.2 to 4.0 | 0.4 to 2.0 | 0.5 | 0.5 |
| compound b) | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 71 | 72 | 73 | 74 | 75 |
| Quaternized collagen | 0.05 to 4.0 | 0.1 to 2.0 | 0.2 to 1.0 | 0.3 | 1.0 |
| hydrolysate a) | | | | | |
| Cetyltrimethylammonium | 0.1 to 6.0 | 0.2 to 4.0 | 0.4 to 2.0 | 0.5 | 0.5 |
| chloride | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 76 | 77 | 78 | 79 | 80 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Cetyltrimethylammonium | 0.1 to 6.0 | 0.2 to 4.0 | 0.4 to 2.0 | 0.5 | 0.5 |
| chloride | | | | | |
| Vinylpyrrolidone/vinyl | 0.5 to 12 | 1.0 to 10 | 2.0 to 8.0 | 5.0 | 2.0 |
| acetate copolymer | | | | | |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 81 | 82 | 83 | 84 | 85 |
| Quaternized collagen | 0.05 to 4.0 | 0.1 to 2.0 | 0.2 to 1.0 | 0.3 | 1.0 |
| hydrolysate a) | | | | | |
| Quaternary ammonium | 0.1 to 6.0 | 0.2 to 4.0 | 0.4 to 2.0 | 0.5 | 0.5 |
| compound b) | | | | | |
| Film-forming polymer | 0.5 to 12 | 1.0 to 10 | 2.0 to 8.0 | 5.0 | 2.0 |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 86 | 87 | 88 | 89 | 90 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Quaternary ammonium | 0.1 to 6.0 | 0.2 to 4.0 | 0.4 to 2.0 | 0.5 | 0.5 |
| compound b) | | | | | |
| Film-forming polymer | 0.5 to 12 | 1.0 to 10 | 2.0 to 8.0 | 5.0 | 2.0 |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 91 | 92 | 53 | 94 | 95 |
| Quaternized collagen | 0.05 to 4.0 | 0.1 to 2.0 | 0.2 to 1.0 | 0.3 | 1.0 |
| hydrolysate a) | | | | | |
| Cetyltrimethylammonium | 0.1 to 6.0 | 0.2 to 4.0 | 0.4 to 2.0 | 0.5 | 0.5 |
| chloride | | | | | |
| Film-forming polymer | 0.5 to 12 | 1.0 to 10 | 2.0 to 8.0 | 5.0 | 2.0 |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 96 | 97 | 98 | 99 | 100 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Cetyltrimethylammonium | 0.1 to 6.0 | 0.2 to 4.0 | 0.4 to 2.0 | 0.5 | 0.5 |
| chloride | | | | | |
| Film-forming polymer | 0.5 to 12 | 1.0 to 10 | 2.0 to 8.0 | 5.0 | 2.0 |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 101 | 102 | 103 | 104 | 105 |
| Quaternized collagen | 0.05 to 4.0 | 0.1 to 2.0 | 0.2 to 1.0 | 0.3 | 1.0 |
| hydrolysate a) | | | | | |
| Quaternary ammonium | 0.1 to 6.0 | 0.2 to 4.0 | 0.4 to 2.0 | 0.5 | 0.5 |
| compound b) | | | | | |
| Vinylpyrrolidone/vinyl | 0.5 to 12 | 1.0 to 10 | 2.0 to 8.0 | 5.0 | 2.0 |
| acetate copolymer | | | | | |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 106 | 107 | 108 | 109 | 110 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Quaternary ammonium | 0.1 to 6.0 | 0.2 to 4.0 | 0.4 to 2.0 | 0.5 | 0.5 |
| compound b) | | | | | |
| Vinylpyrrolidone/vinyl | 0.5 to 12 | 1.0 to 10 | 2.0 to 8.0 | 5.0 | 2.0 |
| acetate copolymer | | | | | |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 111 | 112 | 113 | 114 | 115 |
| Quaternized collagen | 0.05 to 4.0 | 0.1 to 2.0 | 0.2 to 1.0 | 0.3 | 1.0 |
| hydrolysate a) | | | | | |
| Cetyltrimethylammonium | 0.1 to 6.0 | 0.2 to 4.0 | 0.4 to 2.0 | 0.5 | 0.5 |
| chloride | | | | | |
| Vinylpyrrolidone/vinyl | 0.5 to 12 | 1.0 to 10 | 2.0 to 8.0 | 5.0 | 2.0 |
| acetate copolymer | | | | | |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |
| | Formula | Formula | Formula | Formula | Formula |
| | 116 | 117 | 118 | 119 | 120 |
| Quaternized collagen | 0.05 to 4.0 | 0.1 to 2.0 | 0.2 to 1.0 | 0.3 | 1.0 |
| hydrolysate a) | | | | | |
| MW: 2000 and | | | | | |
| 2800 g/mol | | | | | |
| Cetyltrimethylammonium | 0.1 to 6.0 | 0.2 to 4.0 | 0.4 to 2.0 | 0.5 | 0.5 |
| chloride | | | | | |
| Vinylpyrrolidone/vinyl | 0.5 to 12 | 1.0 to 10 | 2.0 to 8.0 | 5.0 | 2.0 |
| acetate copolymer | | | | | |
| Silk protein hydrolysate | 0.05 to 4.0 | 0.1 to 2.0 | 0.2 to 1.0 | 0.5 | 0.3 |
| Water | 70 to 99 | 75 to 95 | 80 to 90 | 87 | 85 |
| Ethanol | 1.0 to 10 | 2.0 to 8.0 | 3.0 to 6.0 | 5.0 | 5.0 |
| Misc. | ad 100 | ad 100 | ad 100 | ad 100 | ad 100 |

The cosmetic compositions according to the invention may contain further auxiliary substances, conditioning substances and additives. The proportion by weight of the further ingredients present in the compositions according to the invention in addition to components a) to d) and the optionally added solvents, in particular of the further auxiliary substances, conditioning substances and additives present in said compositions, in the total weight of the compositions according to the invention preferably amounts to less than 10 wt. %, preferably less than 5.0 wt. %, more preferably less than 2.0 wt. % and in particular less than 1.0 wt. %. The proportion by weight of these auxiliary substances, conditioning substances and additives in the total weight of the cosmetic agents according to the invention may for example amount to 0.001 to 2 wt. %, in particular 0.01 to 0.5 wt. %.

The composition according to the invention may contain a protein hydrolysate and/or one of the derivatives thereof as a further conditioning substance. Protein hydrolysates are product mixtures which are obtained by acidically, basically or enzymatically catalyzed degradation of proteins. According to the invention, the term protein hydrolysates also covers total hydrolysates and individual amino acids and the derivatives thereof and mixtures of different amino acids. The molecular weight of the protein hydrolysates which may be used according to the invention is between 75, the molecular weight of glycine, and 200,000, the molecular weight preferably amounting to 75 to 50,000 and particularly preferably to 75 to 20,000 daltons.

Animal protein hydrolysates are for example silk protein hydrolysates which may also assume salt form. They are distributed for example under the trade name “Promois® (Interorgana)”. A temperature-stable animal protein hydrolysate which is preferred according to the invention is Promois Silk 1000. Plant protein hydrolysates are for example wheat protein hydrolysates which are plant protein hydrolysates which are preferred according to the invention.

The hair treatment agents according to the invention may be formulated in conventional application forms, such as for example as a spray solution, as an aerosol spray, as a mousse or shakable lotion. Formulation as a spray solution is preferred according to the invention.

As described above, the cosmetic compositions according to the invention are suitable as heat-protection agents for keratinic fibers, in particular human hair, in the course of heat-assisted deformation methods. The compositions are distinguished by reduced odor formation and, in hair straightener applications, surprisingly furthermore bring about distinctly improved durability of the straightening in comparison with thermal styling sprays known from the prior art. The same applies to the use thereof in connection with curling hair using curling tongs.

The present application therefore also provides the use of a composition according to the invention as a heat-protection agent for keratinic fibers which, in the course of a deformation method, are subjected to heat treatment with temperatures of 50° C. to 350° C., preferably of 80° C. to 300° C., more preferably of 120° C. to 260° C. and in particular of 150° C. to 220° C.

Use of a composition according to the invention as a straightening agent for keratinic fibers which, in the course of a deformation method, are subjected to heat treatment with temperatures of 50° C. to 350° C., preferably of 80° C. to 300° C., more preferably of 120° C. to 260° C. and in particular of 150° C. to 220° C., is furthermore claimed.

The present application further provides a method for temporarily deforming keratinic fibers, preferably human hair, in the course of which the keratinic fibers are subjected to a heat treatment with temperatures of 50° C. to 350° C., preferably of 80° C. to 300° C., more preferably of 120° C. to 260° C. and in particular of 150° C. to 220° C., characterized in that the keratinic fibers are exposed to a cosmetic composition according to the invention before the heat treatment. The keratinic fibers are preferably exposed by being sprayed with the compositions according to the invention.

### EXAMPLES

The following spray solutions were produced (values stated in wt. %):

| | A | B |
|---|---|---|
| VP/VA copolymer¹⁾ | 5.0 | 5.0 |
| Gluadin WQ PP²⁾ | 1.0 | — |
| Lamequat L³⁾ | — | 1.0 |
| Sodium benzoate | 0.1 | 0.1 |
| Genamin CTAC⁴⁾ | 0.5 | 0.5 |
| Water, deionized | 87.87 | 87.87 |
| Ethanol | 5.0 | 5.0 |
| Phosphoric acid, 85% | 0.03 | 0.03 |
| Promois Silk 1000⁵⁾ | 0.5 | 0.5 |

¹⁾Vinylpyrrolidone-vinyl acetate copolymer, stabilized with 0.05 wt % dodecyltrimethylammonium chloride; INCI name: VP/VA Copolymer
²⁾INCI name Lauryldimonium Hydroxypropyl Hydrolyzed Wheat Protein (approx. 35 wt % active substance in water)
³⁾INCI name Lauryldimonium Hydroxypropyl Hydrolyzed Collagen (approx. 35 wt % active substance in water);
⁴⁾Trimethylhexadecylammonium chloride (approx. 29 wt % active substance in water); INCI name: Cetrimonium Chloride
⁵⁾Silk protein hydrolysate; INCI name: Hydrolyzed Silk

When used in heat-assisted hair deformation methods, composition B according to the invention is distinguished by improved heat protection and improved cosmetic properties in comparison with composition A.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition containing
a) at least one first quaternary ammonium compound from the group of quaternized collagen hydrolysates;
b) at least one second quaternary ammonium compound which differs from the first quaternary ammonium compound
c) at least one film-forming polymer
d) water.

2. The cosmetic composition according to claim 1, wherein the first quaternary ammonium compound comprises 0.05 wt % to 4.0 wt % of the total composition.

3. The cosmetic composition according to claim 1, wherein the first quaternary ammonium compound comprises 0.1 wt % to 2.0 wt % of the total composition.

4. The cosmetic composition according to claim 1, wherein the first quaternary ammonium compound comprises 0.2 wt % to 1.0 wt % of the total composition.

5. The cosmetic composition according claim 1, wherein the first quaternary ammonium compound has a molecular weight of above 1000 g/mol.

6. The cosmetic composition according to claim 1, wherein the first quaternary ammonium compound has a molecular weight of 1200 g/mol and 10000 g/mol.

7. The cosmetic composition according to claim 1, wherein the first quaternary ammonium compound has a molecular weight of 1500 g/mol and 5000 g/mol.

8. The cosmetic composition according to claim 1, wherein the first quaternary ammonium compound has a molecular weight of 2000 g/mol and 2800 g/mol.

9. The cosmetic composition according to claim 1, wherein the second quaternary ammonium compound comprises 0.1 wt % to 6.0 wt % of the total composition.

10. The cosmetic composition according to claim 1, wherein the second quaternary ammonium compound comprises 0.2 wt % to 4.0 wt % of the total composition.

11. The cosmetic composition according to claim 1, wherein the second quaternary ammonium compound comprises 0.4 wt % to 2.0 wt % of the total composition.

12. The cosmetic composition according to claim 1, wherein the second quaternary ammonium compound is selected from the group consisting of alkyltrimethylammonium halides, dialkyldimethylammonium halides and trialkylmethylammonium halides.

13. The cosmetic composition according to claim 1, wherein the second quaternary ammonium compound is an alkyltrimethylammonium chlorides.

14. The cosmetic composition according to claim 1, wherein the quaternary ammonium compound is cetyltrimethylammonium chloride.

15. The cosmetic composition according to claim 1, wherein the film-forming polymer comprises 0.5 wt % to 12 wt % of the total composition.

16. The cosmetic composition according to claim 1, wherein the film-forming polymer comprises 1.0 wt % to 10 wt % of the total composition.

17. The cosmetic composition according to claim 1, wherein the film-forming polymer is selected from the group consisting of vinylpyrrolidone/methylimidazolinium chloride copolymers, vinylpyrrolidone/vinyl acetate copolymers, cationic terpolymers of vinylpyrrolidone, dimethylaminopropylmethacrylamide and lauryldimethylpropylmethacrylamidoammonium chloride.

18. The cosmetic composition according to claim 1, wherein water comprises 70 wt % to 99 wt % of the total composition.

19. The cosmetic composition according to claim 1, wherein water comprises 75 wt % to 95 wt % of the total composition.

20. A method for temporarily deforming keratinic fibers, preferably human hair, in the course of which the keratinic fibers are subjected to a heat treatment with temperatures of 50° C. to 350° C., characterized in that the keratinic fibers are exposed to the cosmetic composition according to claim 1 before the heat treatment.
